(19) United States
(12) Patent Application Publication
(10) Pub. No.: US 2024/0209445 A1
(43) Pub. Date: Jun. 27, 2024

(54) A CONTINUOUS METRIC TO MEASURE ENDOTYPE AND CORTICOSTEROID INTERACTION IN SEPTIC SHOCK

(71) Applicants: CHILDREN'S HOSPITAL MEDICAL CENTER, Cincinnati, OH (US); VANDERBILT UNIVERSITY, Nashville, TN (US)

(72) Inventors: Hector WONG, Cincinnati, OH (US); Christopher LINDSELL, Nashville, TN (US)

(73) Assignees: CHILDREN'S HOSPITAL MEDICAL CENTER, Cincinnati, OH (US); VANDERBILT UNIVERSITY, Nashville, TN (US)

(21) Appl. No.: 18/264,741

(22) PCT Filed: Feb. 16, 2022
(86) PCT No.: PCT/US2022/016642

§ 371 Date: Aug. 8, 2023

Related U.S. Application Data
(60) Provisional application No. 63/149,744, filed on Feb. 16, 2021.

(51) Int. Cl. C12Q 1/6883; A61K 31/573; A61K 45/06; G16H 10/40; G16H 50/30

(52) U.S. Cl. CPC C12Q 1/6883; A61K 31/573; A61K 45/06; G16H 10/40; G16H 50/30; C12Q 2600/118; C12Q 2600/158

(57) ABSTRACT

Methods and compositions disclosed herein generally relate to methods of identifying, validating, and measuring clinically relevant, quantifiable biomarkers of diagnostic and therapeutic responses for blood, vascular, cardiac, and respiratory tract dysfunction, particularly as those responses relate to septic shock in patients, such as pediatric. In particular, the invention relates to analyzing biomarkers associated with septic shock in pediatric patients, obtaining a sample from a patient having at least one indication of septic shock, then determining the gene expression mosaic for the patient, wherein the gene expression mosaic can correlate with a predicted outcome and can inform treatment strategy.

### CROSS REFERENCE TO RELATED APPLICATIONS

The present application is a 35 U.S.C. § 371 national phase application from PCT/US2022/016642, A CONTINUOUS METRIC TO MEASURE ENDOTYPE AND CORTICOSTEROID INTERACTION IN SEPTIC SHOCK, filed Feb. 16, 2022, which claims the benefit of priority under 35 U.S.C. § 119(e) to U.S. Provisional Application No. 63/149,744, A CONTINUOUS METRIC TO ASSESS THE INTERACTION BETWEEN ENDOTYPE ASSIGNMENT AND CORTICOSTEROID RESPONSIVENESS IN SEPTIC SHOCK, filed on Feb. 16, 2021.

### STATEMENT REGARDING FEDERALLY-SPONSORED RESEARCH

This invention was made with government support under R35GM126943 awarded by the National Institutes of Health (NIH). The government has certain rights in the invention.

### FIELD OF THE INVENTION

The invention disclosed herein generally relates to the identification and validation of clinically relevant, quantifiable biomarkers of diagnostic and therapeutic responses for blood, vascular, cardiac, and respiratory tract dysfunction, in particular septic shock.

### BACKGROUND

Septic shock and severe sepsis represent a major public health problem in the United States, despite the development of increasingly powerful antibiotics and advanced forms of intensive care unit-based support modalities (see, e.g., Shanley, T. et al. Sepsis, 3rd Ed., St. Louis, MO, Mosby (2006)). Worldwide, septic shock affects millions of adults, killing approximately one in four (see, e.g., Dellinger, R. et al. Crit. Care Med. 36:296-327 (2008)). One study suggests that the incidence and the mortality rates of septic shock in adults are increasing in the United States (Dombrovskiy, V. et al. Crit. Care Med. 35:1244-50 (2007)).

Septic shock is also a major problem in the pediatric age group, as there are ˜42,000 cases of pediatric septic shock per year in the United States alone, with a mortality rate of ˜10% (see, e.g., Watson, R. et al. Am. J. Respir. Crit. Care Med. 167:695-701 (2003)). While the pediatric mortality rate is lower than that of adults, it nonetheless translates to more than 4,000 childhood deaths per year and countless years of lost productivity due to death at a young age. While this high number of pediatric deaths per year from septic shock indicates that more children die per year in the United States from septic shock as the primary cause than those children who die from cancer, funding specifically targeted toward pediatric septic shock is substantially lower than that for pediatric cancer.

Most forms of critical illness reflect heterogeneous syndromes rather than distinct diseases. Septic shock is a clinical syndrome with substantial clinical and biological heterogeneity. Reliable risk stratification of patients with septic shock has numerous clinical applications, but is a challenging task due to the significant patient heterogeneity. Clinical care is challenging because not all therapies are appropriate for all patients. In the absence of differentiating what therapies are best for which patients, outcomes in many critical illnesses have changed incrementally over the last decade. As such, reliable stratification of outcome risk is fundamental to effective clinical practice and clinical research (Marshall J. Leukoc. Biol. 83:471-82 (2008)), as is the ability to differentiate between subclasses of septic shock that have different biology.

### SUMMARY OF THE INVENTION

Embodiments of the invention relate to methods of classifying a patient with septic shock as high risk of adverse outcome and/or mortality or other than high risk of adverse outcome and/or mortality, the method including: obtaining a sample from a pediatric patient with septic shock at a first time point; analyzing the sample to determine the expression levels of 100 biomarkers to generate a gene expression mosaic for the patient; determining a Z value for the patient according to Equation 1:

$$Z = \vert {Ref}_{A} - {Patient}_{i} \vert - \vert {Ref}_{B} - {Patient}_{i} \vert . \qquad Equation 1$$

wherein Ref_{A} is a reference mosaic for endotype A, Ref_{B} is a reference mosaic for endotype B, and Patienti is the gene expression reference mosaic for the patient; classifying the patient as high risk or other than high risk, wherein a classification of high risk includes a Z value below a cutoff Z value, and wherein a classification of other than high risk includes a Z value of the cutoff Z value or greater; and wherein the 100 biomarkers include the biomarkers listed in Table 1.

In some embodiments of the methods, the cutoff Z value can be determined by modeling an interaction between the Z value and receipt of corticosteroids using logistic regression. In some embodiments, the Z value can be fitted using cubic splines.

In some embodiments, the cutoff Z value can be about 15. In some embodiments, the difference between the gene expression reference mosaic for the patient and the reference mosaic can be calculated by pixel-to-pixel intensity difference.

In some embodiments, biomarker expression levels can be determined by mRNA quantification. In some embodiments, biomarker expression levels can be determined by normalized mRNA counts and/or by cycle threshold (CT) values.

In some embodiments, the classification can be combined with one or more patient demographic data and/or clinical characteristics and/or results from other tests or indicia of septic shock. In some embodiments, the patient demographic data and/or clinical characteristics and/or results from other tests or indicia of septic shock can include the septic shock causative organism, the presence or absence or chronic disease, and/or the age, gender, race, and/or co-morbidities of the patient.

In some embodiments, the classification can be combined with one or more additional population-based risk scores. In some embodiments, the one or more population-based risk scores can include Pediatric Sepsis Biomarker Risk Model (PERSEVERE), Pediatric Risk of Mortality (PRISM), Pediatric Index of Mortality (PIM), and/or Pediatric Logistic Organ Dysfunction (PELOD).

In some embodiments, the sample can be obtained within the first hour of presentation with septic shock. In some embodiments, the sample can be obtained within the first 48 hours of presentation with septic shock.

In some embodiments, the methods can further include administering a treatment including one or more corticosteroid to a patient that is not high risk, or administering a treatment including one or more therapy excluding a corticosteroid to a patient that is classified as high risk, to provide a method of treating a pediatric patient with septic shock.

In some embodiments, one or more high risk therapy can be administered to a patient classified as high risk. In some embodiments, the one or more high risk therapy can include immune enhancing therapy, extracorporeal membrane oxygenation/life support, plasmapheresis, pulmonary artery catheterization, and/or high volume continuous hemofiltration. In some embodiments, the immune enhancing therapy can include administration of GMCSF, interleukin-7, and/or anti-PD-1.

Embodiments of the methods can further include improving an outcome in a pediatric patient with septic shock. For example, some embodiments can further include: obtaining a second sample from the treated patient at a second time point; analyzing the second sample to determine the expression levels of the 100 biomarkers listed in claim 1 to generate a gene expression mosaic for the patient; determining the patient's Z value; and maintaining the treatment being administered if the patient's high risk classification has not changed, or changing the treatment being administered if the patient's high risk classification has changed.

In some embodiments, the second time point can be at least 18 hours after the first time point. In some embodiments, the second time point can be in the range of 24 to 96 hours, or longer, after the first time point. In some embodiments, the second time point can be about 1 day, 2 days, 3 days, or longer, after the first time point. In some embodiments, the second time point can be about 2 days after the first time point. In some embodiments, the first time point can be at day 1, wherein day 1 can be within 24 hours of a septic shock diagnosis, and the second time point can be at day 3.

In some embodiments, a patient classified as high risk after the second time point can be administered one or more high risk therapy, which can include, for example, immune enhancing therapy, extracorporeal membrane oxygenation/life support, plasmapheresis, pulmonary artery catheterization, and/or high volume continuous hemofiltration.

In some embodiments, a patient not classified as high risk after the second time point can be administered a treatment comprising one or more corticosteroid. In some embodiments, the patient classified as high risk and administered one or more high risk therapy after the first time point can be not classified as high risk after the second time point.

In some embodiments of the methods, the patient can be a pediatric patient.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1: The left column shows exemplary reference mosaics for endotype A, and the right column shows exemplary reference mosaics for endotype B; the middle column shoes exemplary reference mosaics for endotype A/B. These composite gene expression mosaics for the 100 class-defining genes are based on previous microarray data and represent the mean expression values of the 100 subclass-defining genes within each subclass, where red intensity correlates with increased gene expression and blue intensity correlates with decreased gene expression. The composite mosaics were used as a reference to classify subjects based on NanoString-generated data for the 100 subclass-defining genes. Classification was performed using computer-assisted image analysis. FIG. 1A: exemplary reference mosaics for endotype A (left), endotype A/B (middle), and endotype B (right). FIG. 1B: alternative exemplary reference mosaics for endotype A (left), endotype A/B (middle), and endotype B (right). FIG. 1C: greyscale version of FIG. 1A. FIG. 1D: greyscale version of FIG. 1B.

FIG. 2: The left column shows dot plots depicting the distribution of Z values stratified by 28-day mortality or complicated course, colored by exposure to corticosteroids. The right column shows the logistic models depicting 28-day mortality (or complicated course) as a function of the interaction between corticosteroids and the Z value. For all five logistic regression models, the Z value was fit using cubic splines with three knots and outcome (mortality or complicated course) is plotted as the log₁₀ odds. FIG. 1A: patients in the pediatric derivation cohort (n=425) by 28-day mortality; FIG. 1B: patients in the pediatric validation cohort (n=230) by 28-day mortality; and FIG. 1C: patients in the combined pediatric cohort (n=655) by 28-day mortality; FIG. 1D, patients in the adult validation cohort (VANISH cohort, n=97) by 28-day mortality; and FIG. 1E, patients in the combined pediatric cohort (n=655) by the composite outcome variable, complicated course.

FIG. 3: Dot density plot of Z values according to 28-day mortality and corticosteroid exposure in the combined pediatric cohort (n=655). Horizontal bars represent the respective median Z values. P<0.001, ANOVA on Ranks.

### DETAILED DESCRIPTION OF THE INVENTION

All references cited herein are incorporated by reference in their entirety. Also incorporated herein by reference in their entirety include: U.S. Patent Application No. 61/595,996, BIOMARKERS OF SEPTIC SHOCK, filed on Feb. 7, 2012; U.S. Provisional Application No. 61/721,705, A MULTI-BIOMARKER-BASED OUTCOME RISK STRATIFICATION MODEL FOR ADULT SEPTIC SHOCK, filed on Nov. 2, 2012; International Patent Application No. PCT/US13/25223, A MULTI-BIOMARKER-BASED OUTCOME RISK STRATIFICATION MODEL FOR PEDIATRIC SEPTIC SHOCK, filed on Feb. 7, 2013; International Patent Application No. PCT/US13/25221, A MULTI-BIOMARKER-BASED OUTCOME RISK STRATIFICATION MODEL FOR ADULT SEPTIC SHOCK, filed on Feb. 7, 2013; U.S. Provisional Application No. 61/908,613, TEMPORAL PEDIATRIC SEPSIS BIOMARKER RISK MODEL, filed on Nov. 25, 2013; International Patent Application No. PCT/US14/067438, TEMPORAL PEDIATRIC SEPSIS BIOMARKER RISK MODEL, filed on Nov. 25, 2014; U.S. patent application Ser. No. 15/998,427, SEPTIC SHOCK ENDOTYPING STRATEGY AND MORTALITY RISK FOR CLINICAL APPLICATION, filed on Aug. 15, 2018; U.S. Provisional Application No. 62/616,646, TEMPORAL ENDOTYPE TRANSITIONS REFLECT CHANGING RISK AND TREATMENT RESPONSE IN PEDIATRIC SEPTIC SHOCK, filed on Jan. 12, 2018; International Application No. PCT/US2017/032538, SIMPLIFICATION OF A SEPTIC SHOCK ENDOTYPING STRATEGY FOR CLINICAL APPLICATIONS, filed on May 12, 2017; U.S. Provisional Application No. 62/335,803, SIMPLIFICATION OF A SEPTIC SHOCK ENDOTYPING STRATEGY FOR CLINICAL APPLICATIONS, filed on May 13, 2016; U.S. Provisional Application No. 62/427,778, SIMPLIFICATION OF A SEPTIC SHOCK ENDOTYPING STRATEGY FOR CLINICAL APPLICATIONS, filed on Nov. 29, 2016; U.S. Provisional Application No. 62/428,451, SIMPLIFICATION OF A SEPTIC SHOCK ENDOTYPING STRATEGY FOR CLINICAL APPLICATIONS, filed on Nov. 30, 2016; U.S. Provisional Application No. 62/446,216, SIMPLIFICATION OF A SEPTIC SHOCK ENDOTYPING STRATEGY FOR CLINICAL APPLICATIONS, filed on Jan. 13, 2017; U.S. patent application Ser. No. 16/539,128, SEPTIC SHOCK ENDOTYPING STRATEGY AND MORTALITY RISK FOR CLINICAL APPLICATION, filed on Aug. 13, 2019; and U.S. Provisional Application No. 62/764,831, ENDOTYPE TRANSITIONS DURING THE ACUTE PHASE OF PEDIATRIC SEPTIC SHOCK REFLECT CHANGING RISK AND TREATMENT RESPONSE, filed on Aug. 15, 2018.

Unless otherwise noted, terms are to be understood according to conventional usage by those of ordinary skill in the relevant art.

As used herein, the term “sample” encompasses a sample obtained from a subject or patient. The sample can be of any biological tissue or fluid. Such samples include, but are not limited to, sputum, saliva, buccal sample, oral sample, blood, serum, mucus, plasma, urine, blood cells (e.g., white cells), circulating cells (e.g. stem cells or endothelial cells in the blood), tissue, core or fine needle biopsy samples, cell-containing body fluids, free floating nucleic acids, urine, stool, peritoneal fluid, and pleural fluid, tear fluid, or cells therefrom. Samples can also include sections of tissues such as frozen or fixed sections taken for histological purposes or micro-dissected cells or extracellular parts thereof. A sample to be analyzed can be tissue material from a tissue biopsy obtained by aspiration or punch, excision or by any other surgical method leading to biopsy or resected cellular material. Such a sample can comprise cells obtained from a subject or patient. In some embodiments, the sample is a body fluid that include, for example, blood fluids, serum, mucus, plasma, lymph, ascitic fluids, gynecological fluids, or urine but not limited to these fluids. In some embodiments, the sample can be a non-invasive sample, such as, for example, a saline swish, a buccal scrape, a buccal swab, and the like.

As used herein, “blood” can include, for example, plasma, serum, whole blood, blood lysates, and the like.

As used herein, the term “assessing” includes any form of measurement, and includes determining if an element is present or not. The terms “determining,” “measuring,” “evaluating,” “assessing” and “assaying” can be used interchangeably and can include quantitative and/or qualitative determinations.

As used herein, the term “monitoring” with reference to septic shock refers to a method or process of determining the severity or degree of septic shock or stratifying septic shock based on risk and/or probability of mortality. In some embodiments, monitoring relates to a method or process of determining the therapeutic efficacy of a treatment being administered to a patient.

As used herein, “outcome” can refer to an outcome studied. In some embodiments, “outcome” can refer to 28-day survival/mortality. The importance of survival/mortality in the context of pediatric septic shock is readily evident. The common choice of 28 days was based on the fact that 28-day mortality is a standard primary endpoint for interventional clinical trials involving critically ill patients. In some embodiments, an increased risk for a poor outcome indicates that a therapy has had a poor efficacy, and a reduced risk for a poor outcome indicates that a therapy has had a good efficacy.

As used herein, “outcome” can also refer to resolution of organ failure after 14 days or 28 days or limb loss. Although mortality/survival is obviously an important outcome, survivors have clinically relevant short- and long-term morbidities that impact quality of life, which are not captured by the dichotomy of “alive” or “dead.” In the absence of a formal, validated quality of life measurement tool for survivors of pediatric septic shock, resolution of organ failure can be used as a secondary outcome measure. For example, the presence or absence of new organ failure over one or more timeframes can be tracked.

Patients having organ failure beyond 28 days are likely to survive with significant morbidities having negative consequences for quality of life. Organ failure is generally defined based on published and well-accepted criteria for the pediatric population (Goldstein, B. et al. Pediatr. Crit. Care Med. 6:2-8 (2005)). Specifically, cardiovascular, respiratory, renal, hepatic, hematologic, and neurologic failure can be tracked. In addition, limb loss can be tracked as a secondary outcome. Although limb loss is not a true “organ failure,” it is an important consequence of pediatric septic shock with obvious impact on quality of life.

As used herein, “outcome” can also refer to complicated course. Complicated course as defined herein relates to persistence of two or more organ failures at day seven of septic shock or 28-day mortality.

As used herein, the terms “predicting outcome” and “outcome risk stratification” with reference to septic shock refers to a method or process of prognosticating a patient's risk of a certain outcome. In some embodiments, predicting an outcome relates to monitoring the therapeutic efficacy of a treatment being administered to a patient. In some embodiments, predicting an outcome relates to determining a relative risk of an adverse outcome (e.g. complicated course) and/or mortality. In some embodiments, the predicted outcome is associated with administration of a particular treatment or treatment regimen. Such adverse outcome risk and/or mortality can be high risk, moderate risk, moderate-high risk, moderate-low risk, or low risk. Alternatively, such adverse outcome risk can be described simply as high risk or low risk, corresponding to high risk of adverse outcome (e.g. complicated course) and/or mortality probability, or high likelihood of therapeutic effectiveness, respectively. In some embodiments of the present invention, adverse outcome risk can be determined via the biomarker-based endotyping strategy and/or Z score as described herein. In some embodiments, predicting an outcome relates to determining a relative risk of mortality. Such mortality risk can be high risk, moderate risk, moderate-high risk, moderate-low risk, or low risk. Alternatively, such mortality risk can be described simply as high risk or low risk, corresponding to high risk of death or high likelihood of survival, respectively. As related to the terminal nodes of the decision trees described herein, a “high risk terminal node” corresponds to an increased probability of adverse outcome (e.g. complicated course) and/or mortality according to a particular treatment or treatment regimen, whereas a “low risk terminal node” corresponds to a decreased probability of adverse outcome (e.g. complicated course) and/or mortality according to a particular treatment or treatment regimen.

As used herein, the term “high risk clinical trial” refers to one in which the test agent has “more than minimal risk” (as defined by the terminology used by institutional review boards, or IRBs). In some embodiments, a high risk clinical trial is a drug trial.

As used herein, the term “low risk clinical trial” refers to one in which the test agent has “minimal risk” (as defined by the terminology used by IRBs). In some embodiments, a low risk clinical trial is one that is not a drug trial. In some embodiments, a low risk clinical trial is one that that involves the use of a monitor or clinical practice process. In some embodiments, a low risk clinical trial is an observational clinical trial.

As used herein, the terms “modulated” or “modulation,” or “regulated” or “regulation” and “differentially regulated” can refer to both up regulation (i.e., activation or stimulation, e.g., by agonizing or potentiating) and down regulation (i.e., inhibition or suppression, e.g., by antagonizing, decreasing or inhibiting), unless otherwise specified or clear from the context of a specific usage.

As used herein, the term “subject” refers to any member of the animal kingdom. In some embodiments, a subject is a human patient. In some embodiments, a subject is a pediatric patient. In some embodiments, a pediatric patient is a patient under 18 years of age, while an adult patient is 18 or older.

As used herein, the terms “treatment,” “treating,” “treat,” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” can also encompass delivery of an agent or administration of a therapy in order to provide for a pharmacologic effect, even in the absence of a disease or condition.

As used herein, the term “marker” or “biomarker” refers to a biological molecule, such as, for example, a nucleic acid, peptide, protein, hormone, and the like, whose presence or concentration can be detected and correlated with a known condition, such as a disease state. It can also be used to refer to a differentially expressed gene whose expression pattern can be utilized as part of a predictive, prognostic or diagnostic process in healthy conditions or a disease state, or which, alternatively, can be used in methods for identifying a useful treatment or prevention therapy.

As used herein, the term “expression levels” refers, for example, to a determined level of biomarker expression. The term “pattern of expression levels” refers to a determined level of biomarker expression compared either to a reference (e.g. a housekeeping gene or inversely regulated genes, or other reference biomarker) or to a computed average expression value (e.g. in DNA-chip analyses). A pattern is not limited to the comparison of two biomarkers but is more related to multiple comparisons of biomarkers to reference biomarkers or samples. A certain “pattern of expression levels” can also result and be determined by comparison and measurement of several biomarkers as disclosed herein and display the relative abundance of these transcripts to each other.

As used herein, a “reference pattern of expression levels” refers to any pattern of expression levels that can be used for the comparison to another pattern of expression levels. In some embodiments of the invention, a reference pattern of expression levels is, for example, an average pattern of expression levels observed in a group of healthy or diseased individuals, serving as a reference group.

As used herein, the term “decision tree” refers to a standard machine learning technique for multivariate data analysis and classification. Decision trees can be used to derive easily interpretable and intuitive rules for decision support systems.

The benefit of corticosteroids for septic shock remains unclear, reflecting heterogeneity of treatment effect. The present inventors previously reported a binary septic shock endotype classification based on expression of 100 genes. This binary classification provides information regarding prognosis and corticosteroid responsiveness but does not capture the endotypes continuum and is therefore insufficient.

Herein, a continuous metric for endotype assignment is described, namely the Z value, along with its relationship with septic shock outcomes and corticosteroid responsiveness. In a derivation cohort, a lower Z value was independently associated with increased odds of mortality. When the Z value was fit using cubic splines, the odds of mortality increased considerably as the Z value decreased among patients exposed to corticosteroids. A similar interaction between Z value and corticosteroid exposure was observed when the model was applied to pediatric and adult validation cohorts. The Z value provides more detailed evidence of patients with septic shock in whom corticosteroids are harmful.

### Septic Shock Endotypes

Endotypes represent subgroups of otherwise heterogenous clinical syndromes that share underlying biological commonalities (1-3). Septic shock is the quintessential heterogenous clinical syndrome among critically ill patients. Three research programs independently leveraged the discovery potential of transcriptomics to propose the existence of endotypes among adults with septic shock, defined by shared gene expression patterns broadly corresponding to immunity, inflammation, and coagulation (4-8). The endotypes reported by the respective programs are characterized by differences in clinical features, such as illness severity, organ failure burden, and mortality. More importantly, the respective endotype-defining gene expression signatures lead to differential responses to treatment, including responsiveness to adjunctive corticosteroids (9, 10).

The intrinsic heterogeneity of septic shock implies the existence of distinct subgroups, or endotypes, that can be classified by pathobiological mechanism or treatment response. Studies previously reported by the present inventors have focused on defining and validating gene expression-based endotypes among children with septic shock based on genome-wide expression profiling (11-15). A classification system was derived based on computer-assisted image analysis of gene expression mosaics representing 100 genes, where the strategy relies on a self-organizing map algorithm to generate gene expression mosaics (16, 17) based a panel of 100 genes included in the mosaics and reflecting adaptive immune function and glucocorticoid receptor signaling, which are highly relevant to the pathobiology of septic shock. Endotype A patients have decreased expression of these classifier genes, relative to endotype B patients. The 100-gene signature is provided in Table 1 below.

TABLE 1
List of 100 septic shock subclass-defining genes.

| Gene | | |
|---|---|---|
| Symbol | Genbank | Description |
| APAF1 | NM_013229 | apoptotic peptidase activating factor 1 |
| ARPC5 | AL516350 | actin related protein 2/3 complex, subunit 5, 16 kDa |
| ASAH1 | BC016828 | N-acylsphingosine amidohydrolase (acid ceramidase) 1 |
| ATP2B2 | U15688 | ATPase, Ca++ transporting, plasma membrane 2 |
| BCL6 | NM_001706 | B-cell CLL/lymphoma 6 |
| BMPR2 | AL046696 | bone morphogenetic protein receptor, type II (serine/threonine kinase) |
| BTK | NM_000061 | Bruton agammaglobulinemia tyrosine kinase |
| CAMK2D | AA777512 | calcium/calmodulin-dependent protein kinase (CaM kinase) II delta |
| CAMK2G | AA284757 | calcium/calmodulin-dependent protein kinase (CaM kinase) II gamma |
| CAMK4 | AL529104 | calcium/calmodulin-dependent protein kinase IV |
| CASP1 | AI719655 | caspase 1, apoptosis-related cysteine peptidase (interleukin 1, beta, convertase) |
| CASP2 | AU153405 | caspase 2, apoptosis-related cysteine peptidase |
| CASP4 | U25804 | caspase 4, apoptosis-related cysteine peptidase |
| CASP8 | BF439983 | caspase 8, apoptosis-related cysteine peptidase |
| CD247 | J04132 | CD247 molecule |
| CD3E | NM_000733 | CD3e molecule, epsilon (CD3-TCR complex) |
| CD3G | NM_000073 | CD3g molecule, gamma (CD3-TCR complex) |
| CD79A | M74721 | CD79a molecule, immunoglobulin-associated alpha |
| CREB1 | NM_004379 | cAMP responsive element binding protein 1 |
| CREB5 | NM_004904 | cAMP responsive element binding protein 5 |
| CSNK1A1 | AV704610 | Casein kinase 1, alpha 1 |
| CTNNB1 | AF130085 | catenin (cadherin-associated protein), beta 1, 88 kDa |
| DAPP1 | NM_014395 | dual adaptor of phosphotyrosine and 3-phosphoinositides |
| DBT | AI632010 | dihydrolipoamide branched chain transacylase E2 |
| EP300 | AI459462 | E1A binding protein p300 |
| FAS | X83493 | Fas (TNF receptor superfamily, member 6) |
| FCGR2A | NM_021642 | Fc fragment of IgG, low affinity IIa, receptor (CD32) |
| FCGR2C | U90939 | Fc fragment of IgG, low affinity IIc, receptor for (CD32) |
| FYN | S74774 | FYN oncogene related to SRC, FGR, YES |
| GK | NM_000167 | glycerol kinase |
| GNAI3 | J03005 | guanine nucleotide binding protein (G protein), alpha inhibiting activity polypeptide 3 |
| HDAC4 | NM_006037 | histone deacetylase 4 |
| HLA-DMA | X76775 | major histocompatibility complex, class II, DM alpha |
| HLA-DOA | AL581873 | major histocompatibility complex, class II, DO alpha |
| ICAM3 | NM_002162 | intercellular adhesion molecule 3 |
| IL1A | NM_000575 | interleukin 1, alpha |
| INPP5D | BC027960 | inositol polyphosphate-5-phosphatase, 145 kDa |
| ITGAM | NM_000632 | integrin, alpha M (complement component 3 receptor 3 subunit) |
| ITGAV | AI093579 | integrin, alpha V (vitronectin receptor, alpha polypeptide, antigen CD51) |
| ITGAX | M81695 | integrin, alpha X (complement component 3 receptor 4 subunit) |
| JAK1 | AL039831 | Janus kinase 1 (a protein tyrosine kinase) |
| JAK2 | NM_004972 | Janus kinase 2 (a protein tyrosine kinase) |
| KAT2B | AV735100 | K(lysine) acetyltransferase 2B |
| LAT2 | AF257135 | linker for activation of T cells family, member 2 |
| LYN | AI356412 | v-yes-1 Yamaguchi sarcoma viral related oncogene homolog |
| MAP2K4 | NM_003010 | mitogen-activated protein kinase kinase 4 |
| MAP3K1 | AA541479 | mitogen-activated protein kinase kinase kinase 1 |
| MAP3K3 | BG231756 | mitogen-activated protein kinase kinase kinase 3 |
| MAP3K5 | NM_005923 | mitogen-activated protein kinase kinase kinase 5 |
| MAP3K7 | NM_003188 | mitogen-activated protein kinase kinase kinase 7 |
| MAP4K1 | BE646618 | mitogen-activated protein kinase kinase kinase kinase 1 |
| MAP4K4 | AL561281 | mitogen-activated protein kinase kinase kinase kinase 4 |
| MAPK1 | AA129773 | mitogen-activated protein kinase 1 |
| MAPK14 | AF100544 | mitogen-activated protein kinase 14 |
| MDH1 | AW952547 | Malate dehydrogenase 1, NAD (soluble) |
| MKNK1 | BC002755 | MAP kinase interacting serine/threonine kinase 1 |
| NCOA2 | AU145806 | Nuclear receptor coactivator 2 |
| NCR3 | AF031136 | natural cytotoxicity triggering receptor 3 |
| NFATC1 | NM_006162 | nuclear factor of activated T-cells, cytoplasmic, calcineurin-dependent 1 |
| PAK2 | AA287921 | p21 protein (Cdc42/Rac)-activated kinase 2 |
| PDPR | BE644918 | pyruvate dehydrogenase phosphatase regulatory subunit |
| PIAS1 | NM_016166 | protein inhibitor of activated STAT, 1 |
| PIK3C2A | AA579047 | Phosphoinositide-3-kinase, class 2, alpha polypeptide |
| PIK3C3 | NM_002647 | phosphoinositide-3-kinase, class 3 |
| PIK3CA | AA767763 | Phosphoinositide-3-kinase, catalytic, alpha polypeptide |
| PIK3CD | U86453 | phosphoinositide-3-kinase, catalytic, delta polypeptide |
| PIK3R1 | AI679268 | phosphoinositide-3-kinase, regulatory subunit 1 (alpha) |
| PLCG1 | AL022394 | phospholipase C, gamma 1 |
| POU2F2 | AA805754 | POU class 2 homeobox 2 |
| PPP1R12A | AI817061 | protein phosphatase 1, regulatory (inhibitor) subunit 12A |
| PPP2R2A | AI934447 | protein phosphatase 2 (formerly 2A), regulatory subunit B, alpha isoform |
| PPP2R5C | AL834350 | protein phosphatase 2, regulatory subunit B′, gamma isoform |
| PRKAR1A | AI682905 | protein kinase, cAMP-dependent, regulatory, type I, alpha (tissue specific extinguisher 1) |
| PRKCB | M13975 | protein kinase C, beta |
| PSMB7 | AI248671 | Proteasome (prosome, macropain) subunit, beta type, 7 |
| PTEN | BC005821 | phosphatase and tensin homolog |
| PTPRC | NM_002838 | protein tyrosine phosphatase, receptor type, C |
| RAF1 | BI496583 | V-raf-1 murine leukemia viral oncogene homolog 1 |
| RHOT1 | NM_018307 | ras homolog gene family, member T1 |
| ROCK1 | N22548 | Rho-associated, coiled-coil containing protein kinase 1 |
| SEMA4F | AF119878 | sema domain, immunoglobulin domain (Ig), transmembrane domain (TM) and short |
| | | cytoplasmic domain, (semaphorin) 4F |
| SEMA6B | NM_020241 | sema domain, transmembrane domain (TM), and cytoplasmic domain, (semaphorin) 6B |
| SMAD4 | AL832789 | SMAD family member 4 |
| SOS1 | AW241962 | son of sevenless homolog 1 (Drosophila) |
| SOS2 | L20686 | son of sevenless homolog 2 (Drosophila) |
| SP1 | BG431266 | Sp1 transcription factor |
| TAF11 | BQ709323 | TAF11 RNA polymerase II, TATA box binding protein (TBP)-associated factor, 28 kDa |
| TBK1 | NM_013254 | TANK-binding kinase 1 |
| TGFBR1 | AV700621 | Transforming growth factor, beta receptor 1 |
| TLE4 | AL358975 | transducin-like enhancer of split 4 (E(sp1) homolog, Drosophila) |
| TLR1 | AL050262 | toll-like receptor 1 |
| TLR2 | NM_003264 | toll-like receptor 2 |
| TLR8 | AW872374 | toll-like receptor 8 |
| TNFSF10 | AW474434 | tumor necrosis factor (ligand) superfamily, member 10 |
| TRA@ | L34703 | T cell receptor alpha locus |
| TYROBP | NM_003332 | TYRO protein tyrosine kinase binding protein |
| UBE3A | AF037219 | Ubiquitin protein ligase E3A |
| USP48 | NM_018391 | ubiquitin specific peptidase 48 |
| ZAP70 | AI817942 | zeta-chain (TCR) associated protein kinase 70 kDa |
| ZDHHC17 | AI621223 | zinc finger, DHHC-type containing 17 |

Mosaics from individual patients are compared to the reference mosaics using computer assisted image analysis. The Z value computed when comparing the individual mosaics to the references was used as a binary classifier. A negative value defined endotype A, while a positive value defined endotype B. This binary classification has direct potential for precision medicine by broadly identifying likely treatment response to adjunctive corticosteroids.

Pediatric endotype A patients are characterized by a higher mortality rate and greater organ failure burden when compared to endotype B patients, and corticosteroid prescription is associated with increased risk of mortality among endotype A patients after adjusting for illness severity (14). Conversely, among the subset of endotype B patients who are at intermediate to high baseline risk of mortality, as measured by the Pediatric Sepsis Biomarker Risk Model (18), corticosteroid prescription is associated with decreased risk of poor outcome after adjusting for illness severity (19).

The clinical utility of molecular endotyping is not in prognostication per se. Rather, the primary clinical utility is in the identification of septic shock subgroups based on biological differences having the potential to inform therapeutic decisions beyond antibiotics and supportive care. Two potential therapies relevant to the endotyping strategy are corticosteroids and immune modulation. The endotyping strategy is based on genes directly involved in the biological pathways targeted by corticosteroids and immune modulation. For example, patients who persist as endotype A are the best candidates for immune enhancing therapies, and corticosteroids should be avoided in such patients. Conversely, previous studies by the present inventors indicate that endotype B patients who are at higher baseline risk of mortality, might derive the most benefit from adjunctive corticosteroids.

The current pediatric endotyping strategy assumes a dichotomy, but given the complexity and heterogeneity of critical illness, pediatric septic shock endotypes can exist along a biological continuum. To develop a more complete understanding of the relationships between endotype assignment and response to corticosteroids, and further inform treatment decisions, the endotype Z value was evaluated as a continuous metric. Such a continuous metric can more accurately capture the endotype continuum and non-arbitrary cutoff points of the Z value are evident. Accordingly, the present invention demonstrates the relationship between septic shock outcomes and endotype Z value, and provides evidence for the role of the Z value in further describing differential treatment response to corticosteroids among children with septic shock.

The role of adjunctive corticosteroids in septic shock has been intensely debated since the 1960s (26). The rationale for prescribing corticosteroids for patients with septic shock reflects their pluripotent effects on inflammation, immunity, and the cardiovascular system. Across multiple, large, randomized clinical trials testing the efficacy of adjunctive corticosteroids among adults with septic shock, there is the consistent observation that corticosteroids reduce vasopressor requirements, but there has not been a consistent demonstration of a survival benefit (27-30). Among children with septic shock, the efficacy of adjunctive corticosteroids is less clear given the lack of comparable data from large, randomized trials (31).

It has been suggested that the inability of corticosteroids to consistently confer a survival benefit in septic shock reflects heterogeneity of treatment effect among subgroups of patients enrolled in clinical trials, such that any potential survival benefit is attenuated by the inclusion of patients in whom corticosteroids are of no benefit or harmful (1, 3, 32, 33). Further, it has been suggested that defining sepsis endotypes provides an opportunity to identify which subgroups of patients with septic shock are more likely to respond favorably, or unfavorably, to adjunctive corticosteroids. This latter concept is well supported by observational studies that employed a binary endotyping strategy based on whole blood-derived gene expression patterns (9, 10, 14).

The current study evaluates a continuous metric to better understand heterogeneity of treatment effect with respect to adjunctive corticosteroids and septic shock. The rationale for this approach is that complex critical illnesses, such as septic shock, can reflect a continuum of underlying biology that cannot be fully captured by a binary designation of, for example, endotype A vs. endotype B. A measure capturing this continuum can enable more precise treatment decisions.

The Z value is a measure of how different an individual's gene expression mosaic is from a reference, and it can be used to approximate where an individual patient resides along the continuum of endotype A to endotype B. In the original endotyping strategy, any negative Z value would have yielded an endotype A assignment, and endotype A patients had increased odds of mortality, independent of baseline illness severity and corticosteroid exposure. Consistent with this previous observation, lower Z values are independently associated with increased odds of mortality. Among patients in the derivation cohort who were exposed to corticosteroids, the odds of mortality substantially increased as the Z value decreased below 15, whereas the odds of mortality were relatively constant with Z values greater than 15. This association was corroborated in a new, previously unreported cohort of children with septic shock, and in a cohort of adults who were enrolled in the VANISH trial. Sensitivity analyses based on the combined cohort and using an alternative outcome variable, further supported these observations.

In the two pediatric cohorts, corticosteroid prescription was at the discretion of the clinical team caring for the patient. Accordingly, corticosteroid prescription was neither random, nor standard, as would occur in a clinical trial. In order to account for illness severity, wherein it is possible that corticosteroid prescription could be more likely among patients having greater illness severity, PRISM scores were included in the logistic regression model.

Collectively, these data provide further and more detailed evidence that there likely exist patients with septic shock in whom adjunctive corticosteroids are more likely to cause harm. This contingency is potentially a major impediment to the conduct of clinical trials focused on adjunctive corticosteroids for septic shock and must be taken into consideration in the design of future trials. Gene expression-based endotyping strategies, such as the one described herein, provide a potential strategy for addressing this challenge (35). A continuous metric can enable more precise treatment decisions, compared to a binary classifier. Of note, two recently launched trials of corticosteroids among children and adults with septic shock are incorporating gene expression-based endotyping strategies into their respective study designs as a means of identifying which patients with septic shock will benefit from adjunctive corticosteroids (NCT03401398 and NCT04280497; clinicaltrials<dot>gov).

Assigning patients to an endotype can enable precision critical care medicine, as identification of these endotypes can identify patients who are more likely to respond and/or who are candidates for to a certain type of treatment, such as, for example, immune enhancing therapies or corticosteroids, and the like. As described herein, the present researchers have demonstrated that prescription of corticosteroids was independently associated with increased odds of mortality in one of the two endotypes (5). In a separate analysis, endotype assignment was combined with risk stratification to identify a subgroup of patients in whom prescription of corticosteroids was associated with improved outcomes (6).

As different subclasses of septic shock have been identified to have different biological activity, with respect to adaptive immunity and glucocorticoid receptor signaling, and differential response to corticosteroid administration, patients classified into the subgroup of patients with increased risk of adverse outcome when prescribed corticosteroids, i.e. endotype A patients, can have improved outcomes when treated with non-corticosteroid therapies. Such non-corticosteroid therapies can include alternative therapies and/or high risk therapies. In particular, endotype A patients can be treated with immune enhancing therapies, such as, for example, GMCSF, interleukin-7, anti-PD-1, and the like.

### Endotype Transitions

Previous studies have measured the 100 endotyping genes at day 1 and day 3 of illness to determine if endotype assignment changes over time, and whether changing endotype is associated with corticosteroid response and outcomes. Corticosteroids were associated with increased risk of mortality among subjects who persisted as endotype A. Therefore, a substantial proportion of children with septic shock were found to transition endotypes during the acute phase of illness. The risk of poor outcome and the response to corticosteroids change with changes in endotype assignment. Patients persisting as endotype A are at highest risk of poor outcomes. This study is described in Wong et al., Crit Care Med, 46:e242-e249, March 2018 (incorporated by reference in its entirety, and for all purposes).

Previous work by the present inventors have demonstrated that a substantial proportion of children with septic shock transition endotypes over the first 3 days of illness. The risk of mortality is most strongly associated with the day 1 endotype, but is modified by the day 3 endotype. Corticosteroids are associated with poor outcomes among patients with a persistent endotype A, but not in those who transition from endotype A to B, nor in those initially assigned to endotype B. Given that the biology associated with the endotype-defining genes, the effects of these endotype transitions on septic shock outcomes and treatment responses warrant further studies.

This Z score can therefore be used to stratify patients, to determine an appropriate therapy, or to monitor the therapeutic efficacy of a treatment being administered to a patient with septic shock. This Z score can be used as an adjunct to physiological assessment for selecting an appropriate therapeutic intervention or monitoring the efficacy of a therapeutic intervention in children with septic shock, where risk of adverse outcome is minimized, or to serve as a surrogate outcome variable in clinical trials. The treatment strategy can be modified after an initial determination and therapeutic intervention, by determining a patient's Z score at multiple time points, e.g. a first time point and at one or more later time point which is some time after the first time point. For example, the first time point can be an initial time point within the first 24 hours of a septic shock diagnosis, with an appropriate therapeutic intervention based on the patient Z score at the initial time point. A patient's Z score can then be determined at one or more later time points, with the therapeutic intervention being maintained or modified as appropriate based on any changes (or lack thereof) to the patient endotype. Any changes to patient Z score can continue to be determined, with the therapeutic intervention being maintained or modified as appropriate based on any changes (or lack thereof) to the patient Z score, over the disease course, at multiple later time points. Later time points can be about 24 hours or longer after the first time point, or about 24 hours or longer after the preceding time point.

### Determining Mortality Risk

Reliable risk stratification has numerous clinical applications. These include better-informed allocation of critical care resources, appropriate selection of patients for higher risk and more costly therapies, and for benchmarking outcomes. Additionally, risk stratification can serve as a prognostic enrichment tool to greatly enhance efficiency of clinical trials. Reliable risk stratification of patients with septic shock can be a challenging task due to significant patient heterogeneity.

The Pediatric Sepsis Biomarker Risk Model (PERSEVERE) for estimating baseline mortality risk in children with septic shock was previously derived and validated. PERSEVERE is based on a panel of 12 serum protein biomarkers measured from blood samples obtained during the first 24 hours of a septic shock diagnosis, selected from among 80 genes having an association with mortality risk in pediatric septic shock.

The PERSEVERE biomarkers were initially identified through discovery-oriented transcriptomic studies searching for genes having an association with mortality in pediatric septic shock. From among the 80 genes identified in these studies, the biomarkers to be considered for inclusion in PERSEVERE were selected using two simultaneous criteria. First, the gene should have a biologically plausible link to septic shock pathophysiology. Second, the protein transcribed from the gene can be readily measured in the blood compartment. While pragmatic, the selection criteria were limited by existing knowledge and paradigms of septic shock pathophysiology, and by technical considerations, leaving just 12 potential biomarkers for consideration. Consequently, 68 genes were left unconsidered, some of which might have the ability to improve upon the ability of PERSEVERE to estimate baseline mortality risk, and some of which might provide information about biological mechanisms and pathophysiology associated with mortality in septic shock.

### Additional Patient Information

The demographic data, clinical characteristics, and/or results from other tests or indicia of septic shock specific to a pediatric patient with septic shock can affect the patient's outcome risk. Accordingly, such demographic data, clinical characteristics, and/or results from other tests or indicia of septic shock can be incorporated into the methods described herein which allow for stratification of individual pediatric patients in order to determine the patient's outcome risk. Such demographic data, clinical characteristics, and/or results from other tests or indicia of septic shock can also be used in combination with the methods described herein which allow for stratification of individual pediatric patients in order to determine the patient's outcome risk.

Such pediatric patient demographic data can include, for example, the patient's age, race, gender, and the like. In some embodiments, the biomarker-based endotyping strategy and/or the mortality probability stratification strategy described herein can incorporate or be used in combination with the patient's age, race, and/or gender to determine an outcome risk.

Such patient clinical characteristics and/or results from other tests or indicia of septic shock can include, for example, the patient's co-morbidities and/or septic shock causative organism, and the like.

Patient co-morbidities can include, for example, acute lymphocytic leukemia, acute myeloid leukemia, aplastic anemia, atrial and ventricular septal defects, bone marrow transplantation, caustic ingestion, chronic granulomatous disease, chronic hepatic failure, chronic lung disease, chronic lymphopenia, chronic obstructive pulmonary disease (COPD), congestive heart failure (NYHA Class IV CHF), Cri du Chat syndrome, cyclic neutropenia, developmental delay, diabetes, DiGeorge syndrome, Down syndrome, drowning, end stage renal disease, glycogen storage disease type 1, hematologic or metastatic solid organ malignancy, hemophagocytic lymphohistiocytosis, hepatoblastoma, heterotaxy, hydrocephalus, hypoplastic left heart syndrome, IPEX Syndrome, kidney transplant, Langerhans cell histiocytosis, liver and bowel transplant, liver failure, liver transplant, medulloblastoma, metaleukodystrophy, mitochondrial disorder, multiple congenital anomalies, multi-visceral transplant, nephrotic syndrome, neuroblastoma, neuromuscular disorder, obstructed pulmonary veins, Pallister Killian syndrome, Prader-Willi syndrome, requirement for chronic dialysis, requirement for chronic steroids, retinoblastoma, rhabdomyosarcoma, rhabdosarcoma, sarcoma, seizure disorder, severe combined immune deficiency, short gut syndrome, sickle cell disease, sleep apnea, small bowel transplant, subglottic stenosis, tracheal stenosis, traumatic brain injury, trisomy 18, type 1 diabetes mellitus, unspecified brain tumor, unspecified congenital heart disease, unspecified leukemia, VATER Syndrome, Wilms tumor, and the like. Any one or more of the above patient co-morbidities can be indicative of the presence or absence of chronic disease in the patient.

Septic shock causative organisms can include, for example, Acinetobacter baumannii, Adenovirus, Bacteroides species, Candida species, Capnotyophaga jenuni, Cytomegalovirus, Enterobacter cloacae, Enterococcus faecalis, Escherichia coli, Herpes simplex virus, Human metapneumovirus, Influenza A, Klebsiella pneumonia, Micrococcus species, mixed bacterial infection, Moraxella catarrhalis, Neisseria meningitides, Parainfluenza, Pseudomonas species, Serratia marcescens, Staphylococcus aureus, Streptococcus agalactiae, Streptococcus milleri, Streptococcus pneumonia, Streptococcus pyogenes, unspecified gram negative rods, unspecified gram positive cocci, and the like.

In some embodiments, the biomarker-based strategy of determining Z score as described herein can incorporate the patient's co-morbidities to determine an outcome risk and/or mortality probability. In some embodiments, the biomarker-based strategy of determining Z score as described herein can incorporate the patient's septic shock causative organism to determine an outcome risk and/or mortality probability.

In some embodiments, the biomarker-based strategy of determining Z score as described herein can be used in combination with the patient's co-morbidities to determine an outcome risk and/or mortality probability. In some embodiments, the biomarker-based strategy of determining Z score as described herein can be used in combination with the patient's septic shock causative organism to determine an outcome risk and/or mortality probability.

### PERSEVERE and Other Population-Based Risk Scores

As mentioned previously, the PERSEVERE model for estimating baseline mortality risk in children with septic shock was previously derived and validated. PERSEVERE is based on a panel of 12 serum protein biomarkers measured from blood samples obtained during the first 24 hours of a septic shock diagnosis, selected from among 80 genes having an association with mortality risk in pediatric septic shock. Of those 12 serum biomarkers, the derived and validated PERSEVERE model is based on 5 specific biomarkers, namely CCL3, HSPA1B, IL8, GZMB, and MMP8. PERSEVERE additional takes patient age into account.

The PERSEVERE decision tree has 8 terminal nodes. Of these, 3 terminal nodes of the PERSEVERE decision tree are determined to be low risk/low mortality probability (terminal nodes 2, 4, and 7), while 5 terminal nodes of the PERSEVERE decision tree are determined to be intermediate to high risk/high mortality probability (terminal nodes 1, 3, 5, 6, and 8). In some embodiments, the low risk/low mortality probability terminal nodes have a mortality probability between 0.000 and 0.025, while the intermediate to high risk/high mortality probability terminal nodes have a mortality probability greater than 0.025.

The low mortality probability terminal nodes are associated with: non-elevated levels of CCL3 and HSPA1B, and a non-highly elevated level of IL8; or an elevated level of CCL3, and non-elevated levels of IL8 and MMP8; or elevated levels of CCL3 and IL8, a non-elevated level of GZMB, and a patient age greater than 0.5 years. The intermediate and high mortality probability terminal nodes are associated with: non-elevated levels of CCL3 and HSPA1B, and a non-highly elevated level of IL8; or an elevated level of CCL3, and non-elevated levels of IL8 and MMP8; or elevated levels of CCL3 and IL8, a non-elevated level of GZMB, and a patient age greater than 0.5 years. In some embodiments, an elevated level of CCL3 corresponds to a serum CCL3 concentration greater than 160 μg/ml; an elevated level of HSPAIB corresponds to a serum HSPA1B concentration greater than 3.3 μg/ml; an elevated level of IL8 corresponds to a serum IL8 concentration greater than 507 μg/ml; a highly elevated level of IL8 corresponds to a serum IL8 concentration greater than 829 μg/ml; an elevated level of GZMB corresponds to a serum GZMB concentration greater than 55 μg/ml; and an elevated level of MMP8 corresponds to a serum MMP8 concentration greater than 47.5 ng/ml.

In some embodiments of the present invention, a patient sample is analyzed for the PERSEVERE serum protein biomarkers, as well as for the TP53 mRNA biomarkers.

In some embodiments of the present invention, the PERSEVERE mortality probability stratification can be used in combination with a patient endotyping strategy and/or Z score determination. In some embodiments, the PERSEVERE-XP mortality probability stratification based on the PERSEVERE serum protein biomarkers and the TP53 mRNA biomarkers, as described herein, can be used in combination with a patient endotyping strategy and/or Z score determination. In some embodiments, the combination of a mortality probability stratification, such as PERSEVERE or PERSEVERE-XP, as described herein, with an endotyping strategy and/or Z score determination, can be used to determine an appropriate treatment regimen for a patient. For example, such combinations can be used to identify which patients are more likely to benefit from corticosteroids.

A number of additional models that generate mortality prediction scores based on physiological variables have been developed to date. These can include the PRISM, Pediatric Index of Mortality (PIM), and/pediatric logistic organ dysfunction (PELOD) models, and the like.

Such models can be very effective for estimating population-based outcome risks but are not intended for stratification of individual patients. The methods described herein which allow for stratification of individual patients can be used alone or in combination with one or more existing population-based risk scores.

In some embodiments, the biomarker-based Z score determination of high risk of adverse outcome and/or mortality described herein can be used with one or more additional population-based risk scores. In some embodiments, the biomarker-based Z score determination of high risk of adverse outcome and/or mortality described herein can be used in combination with PRISM. In some embodiments, the biomarker-based Z score determination of high risk of adverse outcome and/or mortality described herein can be used in combination with PIM. In some embodiments, the biomarker-based Z score determination of high risk of adverse outcome and/or mortality described herein can be used in combination with PELOD. In some embodiments, the biomarker-based Z score determination of high risk of adverse outcome and/or mortality described herein can be used in combination with a population-based risk score other than PRISM, PIM, and PELOD.

### High Risk Therapies

High risk, invasive therapeutic and support modalities can be used to treat septic shock. The methods described herein which allow for the patient's outcome risk to be determined can help inform clinical decisions regarding the application of high risk therapies to specific pediatric patients, based on the patient's outcome risk.

High risk therapies include, for example, extracorporeal membrane oxygenation/life support, plasmapheresis, pulmonary artery catheterization, high volume continuous hemofiltration, and the like. High risk therapies can also include non-corticosteroid therapies, e.g. alternative therapies and/or high risk therapies. In particular, endotype A patients can be treated with immune enhancing therapies, such as, for example, GMCSF, interleukin-7, anti-PD-1, and the like.

In some embodiments, individualized treatment can be provided to a pediatric patient by selecting a pediatric patient classified as high risk by the methods described herein for one or more high risk therapies. In some embodiments, individualized treatment can be provided to a pediatric patient by excluding a pediatric patient classified as low risk from one or more high risk therapies.

Certain embodiments of the invention include using quantification data from a gene-expression analysis and/or from a mRNA analysis, from a sample of blood, urine, saliva, broncho-alveolar lavage fluid, or the like. Embodiments of the invention include not only methods of conducting and interpreting such tests but also include reagents, compositions, kits, tests, arrays, apparatuses, processing devices, assays, and the like, for conducting the tests. The compositions and kits of the present invention can include one or more components which enable detection of the biomarkers disclosed herein and combinations thereof and can include, but are not limited to, primers, probes, cDNA, enzymes, covalently attached reporter molecules, and the like.

Diagnostic-testing procedure performance is commonly described by evaluating control groups to obtain four critical test characteristics, namely positive predictive value (PPV), negative predictive value (NPV), sensitivity, and specificity, which provide information regarding the effectiveness of the test. The PPV of a particular diagnostic test represents the proportion of positive tests in subjects with the condition of interest (i.e. proportion of true positives); for tests with a high PPV, a positive test indicates the presence of the condition in question. The NPV of a particular diagnostic test represents the proportion of negative tests in subjects without the condition of interest (i.e. proportion of true negatives); for tests with a high NPV, a negative test indicates the absence of the condition. Sensitivity represents the proportion of subjects with the condition of interest who will have a positive test; for tests with high sensitivity, a positive test indicates the presence of the condition in question. Specificity represents the proportion of subjects without the condition of interest who will have a negative test; for tests with high specificity, a negative test indicates the absence of the condition.

The threshold for the disease state can alternatively be defined as a 1-D quantitative score, or diagnostic cutoff, based upon receiver operating characteristic (ROC) analysis. The quantitative score based upon ROC analysis can be used to determine the specificity and/or the sensitivity of a given diagnosis based upon subjecting a patient to a decision tree described herein in order to predict an outcome for a pediatric patient with septic shock.

The correlations disclosed herein, between pediatric patient septic shock biomarker levels and/or mRNA levels and/or gene expression levels, provide a basis for conducting a diagnosis of septic shock, or for conducting a stratification of patients with septic shock, or for enhancing the reliability of a diagnosis of septic shock by combining the results of a quantification of a septic shock biomarker with results from other tests or indicia of septic shock, or for determining an appropriate treatment regimen for a pediatric patient with septic shock. For example, the results of a quantification of one biomarker could be combined with the results of a quantification of one or more additional biomarker, cytokine, mRNA, or the like. Thus, even in situations in which a given biomarker correlates only moderately or weakly with septic shock, providing only a relatively small PPV, NPV, specificity, and/or sensitivity, the correlation can be one indicium, combinable with one or more others that, in combination, provide an enhanced clarity and certainty of diagnosis. Accordingly, the methods and materials of the invention are expressly contemplated to be used both alone and in combination with other tests and indicia, whether quantitative or qualitative in nature.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Methods Patient Endotyping Strategy

The methods used in Examples 2-6 are summarized below:

### Study Subjects and Data Collection.

The study subjects for deriving a model to describe the interaction between the Z value and corticosteroid prescription were previously reported (14, 19, 20), whereas the study subjects for testing the model were newly enrolled for this study. Briefly, children≤ 18 years of age admitted to the pediatric intensive care unit (PICU) and meeting pediatric-specific criteria for septic shock were enrolled after informed consent from parents or legal guardians (21). Blood samples were obtained within 24 hours of initial presentation to the PICU with septic shock. Total RNA was isolated from whole blood using the PaxGene™ Blood RNA System (PreAnalytiX, Qiagen/Becton Dickson, Valencia, CA). Clinical and laboratory data were collected daily while in the PICU. Mortality and organ failure were tracked for 28 days after enrollment. Complicated course was defined as death by 28 days or persistence of two or more organ failures at day seven of septic shock (19). No patients were censored for reasons other than mortality prior to the 28-day follow-up period. Baseline illness severity was measured using PRISM scores (22).

The model was also tested using a publicly available transcriptomic data set from the Vasopressin vs. Norepinephrine as Initial Therapy for Septic Shock (VANISH) trial (9, 23). The VANISH trial compared the efficacy of vasopressin to norepinephrine as initial vasopressor therapy for septic shock among adults. Patients who reached a prespecified dose of either vasopressor were further randomized to receive hydrocortisone or placebo. Details of the procedures for assigning the cohort randomized to hydrocortisone or placebo to pediatric endotypes A or B were previously described (10, 15).

Multiplex mRNA Quantification and Z Value Calculation.

A custom NanoString nCounter™ codeset (NanoString Technologies, Seattle, WA) was generated for the 100 endotype-defining genes, as previously described (14). Gene expression mosaics representing the expression patterns of the 100 genes were generated using the Gene Expression Dynamics Inspector (GEDI), using four housekeeping genes to normalize the NanoString-derived expression data (B-2-microglobulin (B2M), folylpolyglutamate synthase (FPGS), 2,4-dienoyl CoA reductase 1 (DECR1), and peptidylprolyl isomerase B (PPIB)), based on the geometric mean of the housekeeping genes, as previously described (13, 15-17). The signature graphical outputs of GEDI are expression mosaics that give microarray data a “face” that is intuitively recognizable via human pattern recognition. The algorithm for creating the mosaics is a self-organizing map (13). These reference mosaics represent the average expression patterns of the individual patients representing a given subclass, where the composite reference mosaics represent the mean expression values of the 100 subclass-defining genes within each of endotypes A and B (12). For example, see Wong et al., Developing a clinically feasible personalized medicine approach to pediatric septic shock. Am J Respir Crit Care Med 2015; 191: 309-315.

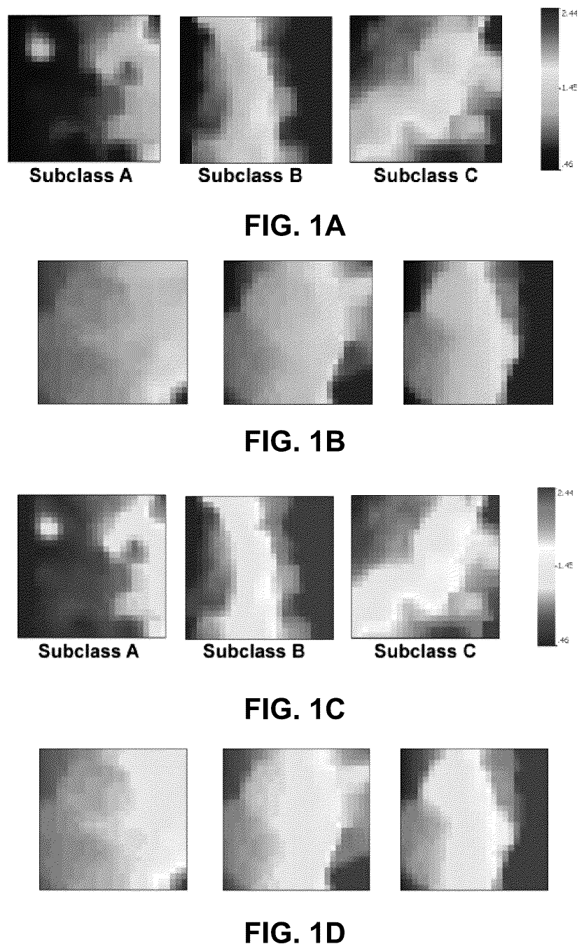

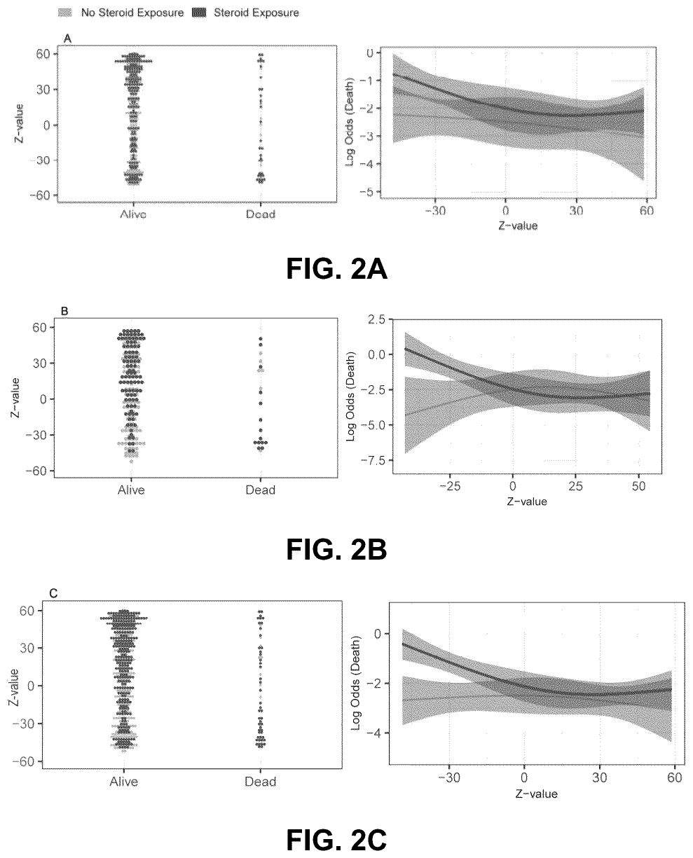

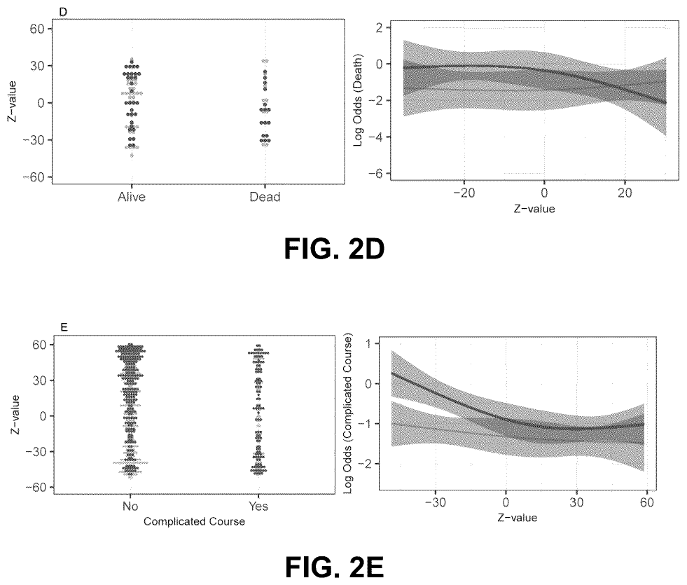

See FIG. 1, which has 2 sets of exemplary endotype A and B reference mosaics (as well as endotype A/B reference mosaics). Exemplary reference mosaics for endotype A and endotype B can also be found at Wong et al., Toward a clinically feasible gene expression-based subclassification strategy for septic shock: proof of concept. Crit Care Med 2010; 38: 1955-1961 (available online at https<colon slash slash>www<dot>ncbi<dot>nlm<dot>nih<dot>gov<slash>labs<slash>pmc<slash>articles<slash>PMC2943553). FIGS. 1A and 1B show exemplary endotype A and B reference mosaics, showing the RGB color variation. FIGS. 1C and 1D show greyscale versions of FIGS. 1A and 1B, respectively. In the gene expression mosaics, red intensity correlates with increased gene expression and blue intensity correlates with decreased gene expression. The gene expression mosaics from individual patients were compared to endotype A and B reference mosaics via computer assisted image analysis using a public analysis platform (ImageJ; National Institutes of Health, Bethesda, MD), as previously described (13). Specifically, the “difference” function in Image J was used, which takes the absolute difference between the study subject and the respective reference mosaic The absolute difference in RGB pixel-to-pixel intensity was calculated for each individual patient mosaic, relative to the reference mosaics representing the septic shock subclasses, e.g. the full color reference mosaics shown in FIG. 1A, or the full color reference mosaics shown in FIG. 2B, which clearly depict the color, including the RGB pixel intensity.

Z values were calculated using the equation:

$$Z = \vert {Ref}_{A} - {Patient}_{i} \vert - \vert {Ref}_{B} - {Patient}_{i} \vert$$

Where Ref_{A} is the reference mosaic for endotype A, Ref_{B} is the reference mosaic for endotype B, and Patienti is the reference mosaic for an individual patient.

### Statistical Analyses.

Demographic and clinical characteristics are summarized using medians, interquartile ranges, frequencies, and percentages. In the derivation dataset, a logistic regression model was fit to predict 28-day mortality. Covariates considered were the Z values, which was modeled using restricted cubic splines with 3 knots, PRISM to adjust for severity, and corticosteroid use. Age and comorbidity burden were also considered as possible predictors of mortality. The interaction between corticosteroid prescription and Z values was evaluated to discern possible differential treatment effects. Post hoc, given the similarity of findings in the derivation and validation of the model, the final model was fit to the combined datasets using the same model specification to provide more precise estimates of the observed effects. Analyses used SPSS 27.0 (IBM Corporation, Armonk, NY) and R (package rms) (24, 25).

### Example 2

### Demographic and Clinical Data

Table 2 shows the demographic and clinical data for the derivation cohort (n=425). Forty-eight subjects (11%) did not survive to 28 days. The median Z value for the derivation cohort was 16, with an interquartile range of −29 to 45. FIG. 2A provides the distribution of Z values among the derivation cohort subjects according to 28-day mortality and receipt of corticosteroids.

To characterize the association between Z value and outcomes, logistic regression model was fit to predict 28-day mortality. Lower Z value, higher PRISM score, and corticosteroid exposure were each independently associated with increased odds of 28-day mortality in univariable models, whereas age and comorbidity burden were not (Table 3). A lower Z value remained associated with increased odds of 28-day mortality after adjusting for the PRISM score and corticosteroid exposure, consistent with previously reported associations between binary endotype assignment and 28-day mortality.

TABLE 2
Demographic and clinical data for the pediatric
derivation and validation cohorts.

| | Derivation Cohort | Validation Cohort |
|---|---|---|
| N | 425 | 230 |
| Median age, years | 2.5 (1.0-6.2) | 3.8 (1.3-7.0)¹ |
| Females, n (%) | 177 (42) | 110 (48) |
| PRISM | 12 (8-19) | 10 (5-15)¹ |
| Mortality, n (%) | 48 (11) | 20 (9) |
| Complicated course, n (%) | 124 (29) | 51 (22) |
| Maximum Organ Failures, | 2 (2- 3) | 2 (2-3) |
| median | | |
| Median PICU Length of | 7 (3-14) | 8 (4-14) |
| Stay, days² | | |
| Median PICU Free Days | 19 (8-24) | 19 (9-24) |
| Received Corticosteroids, | 220 (52) | 110 (48) |
| n (%) | | |
| Comorbidity, n (%) | 176 (41) | 127 (55)² |
| Malignancy, n (%) | 32 (8) | 16 (7) |
| Immune suppression, n (%) | 43 (10) | 24 (10) |
| Bone marrow | 17 (4) | 17 (7) |
| transplantation, n (%) | | |
| Gram negative infection, | 84 (20) | 61 (27) |
| n (%) | | |
| Gram positive infection, | 88 (21) | 62 (27) |
| n (%) | | |
| Fungal infection, n (%) | 5 (1) | 2 (1) |
| Viral infection, n (%) | 36 (8) | 28 (12) |
| Negative cultures, n (%) | 147 (35) | 83 (36) |
| Median Z value | 16 (−29-45) | 13.4 (−21.2-38.3) |

¹p < 0.05, Rank Sum test.
²Median values are reported with inter-quartile ranges in parentheses.
²p < 0.05, Chi-square test.

TABLE 3
Univariable and multivariable regression for variables associated
with 28-day mortality in the derivation cohort.

| | Univariable Regression | | | Multivariable Regression | | |
|---|---|---|---|---|---|---|
| | | | P | | | P |
| Variable | OR | 95% C.I. | value | OR | 95% C.I. | value |
| Z value | 0.99 | 0.981-0.998 | 0.013 | 0.989 | 0.981-0.998 | 0.015 |
| PRISM | 1.096 | 1.061-1.113 | <0.001 | 1.095 | 1.058-1.133 | <0.001 |
| Corticosteroids | 2.2 | 1.2-4.3 | 0.014 | 2.3 | 1.2-4.5 | 0.018 |
| Co-morbidity | 0.7 | 0.4-1.3 | 0.23 | — | — | — |
| Age | 0.9 | 0.8-1.0 | 0.178 | — | — | — |

### Example 3

### Relationship Between the Z Value and Response to Corticosteroids

To characterize whether the association between corticosteroids and outcomes from septic shock differ based on the Z value, logistic regression was used to model the interaction between the Z value and receipt of corticosteroids. Rather than assume a linear association, the Z value was fit using cubic splines. FIG. 2A shows the association between Z values and outcomes, and the potential for effect modification of corticosteroids. While the interaction term was not statistically significant, among subjects exposed to corticosteroids the odds of mortality start to increase considerably as the Z value decreases below approximately 15. In contrast, among those not exposed to corticosteroids the odds of mortality gradually increase as the Z value decreases, but throughout the entire range of Z values.

### Example 4

### Validation in a Pediatric Cohort

The above observations were made using the same cohort in which the association between corticosteroids and mortality among endotype A patients was originally reported (14). To determine whether the current findings are reproducible, data were analyzed from a new cohort of previously unreported pediatric patients. Table 2 shows the demographic and clinical characteristics of the validation cohort (n=230). Compared to the derivation cohort, the subjects in the validation cohort were older, had lower median PRISM scores, and a greater proportion had a comorbidity. No other differences were noted. FIG. 2B provides the distribution of Z values among the 216 validation cohort subjects according to 28-day outcome and receipt of corticosteroids.

The associations between Z value, corticosteroids, and outcomes in the validation cohort are shown in FIG. 2B. The model shows strong similarities with the original for patients exposed to corticosteroids, with the odds of 28-day mortality considerably increasing below a Z value of approximately 15. Unlike the original model, however, the Z value was not statistically significantly associated with mortality among those not exposed to corticosteroids. There were only 6 deaths among validation cohort patients not exposed to corticosteroids, and among these only one had a negative Z value, resulting in imprecise estimates of treatment effects at lower Z values. Nonetheless, the model yielded a significant interaction, even after adjusting for illness severity using PRISM (p=0.008 for the main effect, p-0.08 for the non-linear term).

### Example 5

### Sensitivity Analyses

To address the possibility that the individual model estimates generated in the derivation and validation cohorts might reflect an insufficient sample size, the derivation and validation cohorts (n=655) were combined, and the analyses were repeated. FIG. 2C provides the distribution of Z values among the combined cohort subjects according to 232 28-day outcome and receipt of corticosteroids. In this larger cohort, lower Z value, higher PRISM score, corticosteroid exposure, and younger age were each associated with increased odds of 28-day mortality in univariable models (Table 4). A lower Z value remained associated with increased odds of 28-day mortality after adjusting for the PRISM score, corticosteroid exposure, and age, consistent with the initial observations.

The interaction between the Z value and receipt of corticosteroids was statistically significant (p=0.05), after adjustment for PRISM score and age. FIG. 2C shows that among subjects exposed to corticosteroids, there was again a steady increase in the odds of mortality below a Z value of approximately 15.

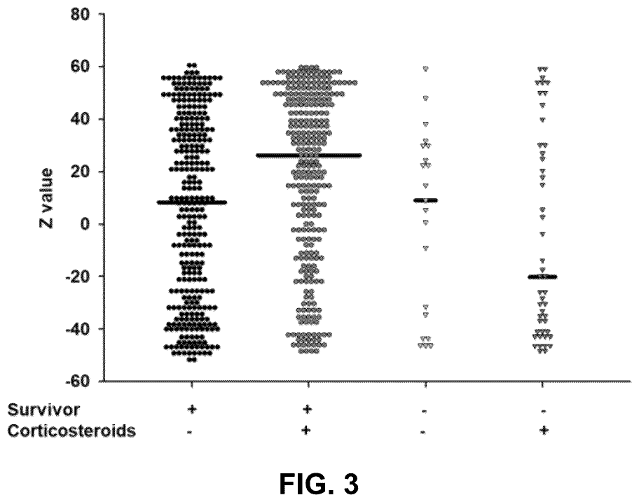

As an additional visualization of the combined cohort data, a dot density plot was generated with median Z values according to 28-day mortality and corticosteroid exposure. As shown in FIG. 3, the median Z value of subjects who received corticosteroids and died by 28 days was significantly lower when compared to the other groups.

TABLE 4
Univariable and multivariable regression for variables associated with 28-
day mortality in the combined derivation and validation pediatric cohorts.

| | Univariable Regression | | | Multivariable Regression | | |
|---|---|---|---|---|---|---|
| | | | P | | | P |
| Variable | OR | 95% C.I. | value | OR | 95% C.I. | value |
| Z Value | 0.988 | 0.981-0.996 | 0.001 | 0.988 | 0.980-0.996 | 0.004 |
| PRISM | 1.112 | 1.081-1.143 | <0.001 | 1.108 | 1.076-1.141 | <0.001 |
| Corticosteroids | 2.4 | 1.4-4.1 | 0.001 | 2.3 | 1.3-4.1 | 0.006 |
| Co-morbidity | 0.7 | 0.4-1.2 | 0.254 | — | — | — |
| Age | 0.9 | 0.8-1.0 | 0.034 | 1 | 0.9-1.1 | 0.987 |

In another sensitivity analysis, complicated course was explored as the outcome variable, instead of 28-day mortality. Complicated course is a composite outcome defined as persistence of multiple organ failure at day seven of septic shock or death by 28 days (14, 19, 20). In univariate models involving the combined cohort, lower Z value, higher PRISM score, corticosteroid exposure, and younger age were each associated with increased odds of complicated course (Table 5).

A lower Z value remained associated with increased odds of complicated course after adjusting for the PRISM score, corticosteroid exposure, and age. FIG. 2E provides the distribution of Z values in the combined cohort according to the complicated course outcome and receipt of corticosteroids.

The interaction between Z value and receipt of corticosteroids was then tested, along with the association with complicated course. FIG. 2E shows that among subjects exposed to corticosteroids, the odds of complicated course steadily increased with decreasing Z score, but the interaction term (corticosteroids x Z score) was not statistically significant after adjusting for PRISM score and age.

TABLE 5
Univariable and multivariable regression for variables associated with complicated
course in the combined derivation and validation pediatric cohorts.

| | Univariable Regression | | | Multivariable Regression | | |
|---|---|---|---|---|---|---|
| | | | P | | | P |
| Variable | OR | 95% C.I. | value | OR | 95% C.I. | value |
| Z Value | 0.933 | 0.988-0.997 | 0.003 | 0.994 | 0.988-1.000 | 0.038 |
| PRISM | 1.098 | 1.074-1.123 | <0.001 | 1.092 | 1.068-1.118 | <0.001 |
| Corticosteroids | 1.7 | 1.2-2.4 | 0.003 | 1.5 | 1.0-2.2 | 0.047 |
| Co-morbidity | 0.8 | 0.5-1.1 | 0.113 | — | — | — |
| Age | 0.9 | 0.8-0.9 | <0.001 | 0.9 | 0.9-1.0 | 0.017 |

### Example 6

### Validation in an Independent Adult Cohort

The findings above were further validated using a subset of adult subjects from the VANISH trial who were randomized to placebo or hydrocortisone, and for whom there existed publicly available transcriptomic data (n=97). FIG. 2D provides the distribution of Z values among the adult validation cohort subjects according to 28-day outcome and receipt of corticosteroids Fitting the same model in this cohort resulted in the associations shown in FIG. 2D. Subjects with no hydrocortisone exposure and lower Z values tended to have lower odds of death at 28 days, compared to those with hydrocortisone exposure and lower Z values, after controlling for illness severity using the APACHE score. The model supports that separation between groups treated with hydrocortisone and those treated with placebo occurs as the Z value decreases.

### Example 7

### Methods of Classifying and Treating Patients Based on Z Score

The biomarkers described herein are used to classify a patient with septic shock as having high risk or low risk of adverse outcome. First, a sample from a patient with septic shock is analyzed to determine the expression levels of 100 biomarkers as listed in Table 1 to generate a gene expression mosaic for the patient. The patient's Z value is then determined according to Equation 1:

$$Z = \vert {Ref}_{A} - {Patient}_{i} \vert - \vert {Ref}_{B} - {Patient}_{i} \vert . \qquad Equation 1$$

wherein Ref_{A} is a reference mosaic for endotype A, Ref_{B} is a reference mosaic for endotype B, and Patienti is gene expression reference mosaic for the patient.

The patient is then classified as high risk of adverse outcome or other than high risk, wherein a classification of high risk comprises a Z value below 15, and wherein a classification of other than high risk comprises a Z value of 15 or greater.

The patient is then administered a treatment based on the patient's classification as high risk or other than high risk. A patient that is classified as other than high risk is administered a treatment which includes one or more corticosteroid, whereas a patient that is classified as high risk is administered a treatment which excludes one or more corticosteroid. A patient that is classified as high risk is then optionally administered a high risk treatment or therapy. In this way, individualized treatment is provided, and clinical outcomes for septic shock patients are improved and morbidity reduced.

A second sample is then obtained, and the patient's classification is confirmed or updated. The treatment being administered is then maintained or revised as necessary. This allows for determination and monitoring of therapeutic efficacy.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about” and/or “approximately.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. As used herein, the terms “about” and “approximately” are used to encompass a deviation or variation of up to about 10%, such as, for example, 1%, 2%, 3%, 4%, 5%, 6%, 7%, 8%, 9%, or 10%, in either direction.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

1. Stanski N L, Wong H R. Prognostic and predictive enrichment in sepsis. Nat Rev Nephrol 2020; 16: 20-31.
2. Prescott H C, Calfee C S, Thompson B T, Angus D C, Liu V X. Toward Smarter Lumping and Smarter Splitting: Rethinking Strategies for Sepsis and Acute Respiratory Distress Syndrome Clinical Trial Design. Am J of Respir Crit Care Med 2016; 194: 147-155.
3 Reddy K, Sinha P, O'Kane C M, Gordon A C, Calfee C S, McAuley D F. Subphenotypes in critical care: translation into clinical practice. Lancet Respir Med 2020; 8: 631-643.
4. Sweeney T E, Azad T D, Donato M, Haynes W A, Perumal T M, Henao R, Bermejo-Martin J F, Almansa R, Tamayo E, Howrylak J A, Choi A, Parnell G P, Tang B, Nichols M, Woods C W, Ginsburg G S, Kingsmore S F, Omberg L, Mangravite L M, Wong H R, Tsalik E L, Langley R J, Khatri P. Unsupervised Analysis of Transcriptomics in Bacterial Sepsis Across Multiple Datasets Reveals Three Robust Clusters. Crit Care Med 2018; 46: 915-925.
5. Davenport E E, Burnham K L, Radhakrishnan J, Humburg P, Hutton P, Mills T C, Rautanen A, Gordon A C, Garrard C, Hill A V, Hinds C J, Knight J C. Genomic landscape of the individual host response and outcomes in sepsis: a prospective cohort study. Lancet Respir Med 2016; 4: 259-271.
6. Scicluna B P, van Vught L A, Zwinderman A H, Wiewel M A, Davenport E E, Burnham K L, Nurnberg P, Schultz M J, Horn J, Cremer O L, Bonten M J, Hinds C J, Wong H R, Knight J C, van der Poll T, consortium M. Classification of patients with sepsis according to blood genomic endotype: a prospective cohort study. Lancet Respir Med 2017; 5: 816-826.
7. Burnham K L, Davenport E E, Radhakrishnan J, Humburg P, Gordon A C, Hutton P, Svoren-Jabalera E, Garrard C, Hill A V S, Hinds C J, Knight J C. Shared and Distinct Aspects of the Sepsis Transcriptomic Response to Fecal Peritonitis and Pneumonia. Am J Respir Crit Care Med 2017; 196: 328-339.
8. Sweeney T E, Liesenfeld O, Wacker J, He Y D, Rawling D, Remmel M, Coyle S, Midic U, Kotsaki A, Kanavou A, Leventogiannis K, Kontogeorgou I, Giamarellos-Bourboulis E J. Validation of Inflammopathic, Adaptive, and Coagulopathic Sepsis Endotypes in Coronavirus Disease 2019. Crit Care Med 2021; 49: e170-e178.
9. Antcliffe D B, Burnham K L, Al-Beidh F, Santhakumaran S, Brett S J, Hinds C J, Ashby D, Knight J C, Gordon A C. Transcriptomic Signatures in Sepsis and a Differential Response to Steroids. From the VANISH Randomized Trial. Am J Crit Care Med 2019; 199: 980-986.
10. Wong H R, Hart K W, Lindsell C J, Sweeney T E. External Corroboration That Corticosteroids May Be Harmful to Septic Shock Endotype A Patients. Crit Care Med 2021; 49: e98-e101.
11. Wong H R, Cvijanovich N, Lin R, Allen G L, Thomas N J, Willson D F, Freishtat R J, Anas N, Meyer K, Checchia P A, Monaco M, Odom K, Shanley T P. Identification of pediatric septic shock subclasses based on genome-wide expression profiling. BMC Med 2009; 7: 34.
12. Wong H R, Wheeler D S, Tegtmeyer K, Poynter S E, Kaplan J M, Chima R S, Stalets E, Basu R K, Doughty L A. Toward a clinically feasible gene expression-based subclassification strategy for septic shock: proof of concept. Crit Care Med 2010; 38: 1955-1961.
13. Wong H R, Cvijanovich N Z, Allen G L, Thomas N J, Freishtat R J, Anas N, Meyer K, Checchia P A, Lin R, Shanley T P, Bigham M T, Wheeler D S, Doughty L A, Tegtmeyer K, Poynter S E, Kaplan J M, Chima R S, Stalets E, Basu R K, Varisco B M, Barr F E. Validation of a gene expression-based subclassification strategy for pediatric septic shock. Crit Care Med 2011; 39: 2511-2517.
14. Wong H R, Cvijanovich N Z, Anas N, Allen G L, Thomas N J, Bigham M T, Weiss S L, Fitzgerald J, Checchia P A, Meyer K, Shanley T P, Quasney M, Hall M, Gedeit R, Freishtat R J, Nowak J, Shekhar R S, Gertz S, Dawson E, Howard K, Harmon K, Beckman E, Frank E, Lindsell C J. Developing a clinically feasible personalized medicine approach to pediatric septic shock. Am J Respir Crit Care Med 2015; 191: 309-315.
15. Wong H R, Sweeney T E, Hart K W, Khatri P, Lindsell C J. Pediatric Sepsis Endotypes Among Adults With Sepsis. Crit Care Med 2017; 45: e1289-e1291.
16. Guo Y, Eichler G S, Feng Y, Ingber D E, Huang S. Towards a holistic, yet gene-centered analysis of gene expression profiles: a case study of human lung cancers. J Biomed Biotechnol 2006; 2006: 69141.
17. Eichler G S, Huang S, Ingber D E. Gene Expression Dynamics Inspector (GEDI): for integrative analysis of expression profiles. Bioinformatics 2003; 19: 2321-2322.
18. Wong H R, Caldwell J T, Cvijanovich N Z, Weiss S L, Fitzgerald J C, Bigham M T, Jain P N, Schwarz A, Lutfi R, Nowak J, Allen G L, Thomas N J, Grunwell J R, Baines T, Quasney M, Haileselassie B, Lindsell C J. Prospective clinical testing and experimental validation of the Pediatric Sepsis Biomarker Risk Model. Sci Transl Med 2019; 11.
19. Wong H R, Atkinson S J, Cvijanovich N Z, Anas N, Allen G L, Thomas N J, Bigham M T, Weiss S L, Fitzgerald J C, Checchia P A, Meyer K, Quasney M, Hall M, Gedeit R, Freishtat R J, Nowak J, Raj S S, Gertz S, Lindsell C J. Combining Prognostic and Predictive Enrichment Strategies to Identify Children With Septic Shock Responsive to Corticosteroids. Crit Care Med 2016; 44: e1000-1003.
20. Wong H R, Cvijanovich N Z, Anas N, Allen G L, Thomas N J, Bigham M T, Weiss S L, Fitzgerald J C, Checchia P A, Meyer K, Quasney M, Hall M, Gedeit R, Freishtat R J, Nowak J, Lutfi R, Gertz S, Grunwell J R, Lindsell C J. Endotype Transitions During the Acute Phase of Pediatric Septic Shock Reflect Changing Risk and Treatment Response. Crit Care Med 2018; 46: e242-e249.
21. Wong H R, Shanley T P, Sakthivel B, Cvijanovich N, Lin R, Allen G L, Thomas N J, Doctor A, Kalyanaraman M, Tofil N M, Penfil S, Monaco M, Tagavilla M A, Odoms K, Dunsmore K, Barnes M, Aronow B J. Genome-level expression profiles in pediatric septic shock indicate a role for altered zinc homeostasis in poor outcome. Physiol Genomics 2007; 30: 146-155.
22. Pollack M M, Holubkov R, Funai T, Dean J M, Berger J T, Wessel D L, Meert K, Berg R A, Newth C J, Harrison R E, Carcillo J, Dalton H, Shanley T, Jenkins T L, Tamburro R, Eunice Kennedy Shriver National Institute of Child H, Human Development Collaborative Pediatric Critical Care Research N. The Pediatric Risk of Mortality Score: Update 2015. Pediatr Crit Care Med 2016; 17: 2-9.
23. Gordon A C, Mason A J, Thirunavukkarasu N, Perkins G D, Cecconi M, Cepkova M, Pogson D G, Aya H D, Anjum A, Frazier G J, Santhakumaran S, Ashby D, Brett S J, Investigators V. Effect of Early Vasopressin vs Norepinephrine on Kidney Failure in Patients With Septic Shock: The VANISH Randomized Clinical Trial. JAMA 2016; 316: 509-518.
24. Team R C. R: A language and environment for statistical computing. R Foundation for Statistical Computing, Vienna, Austria. URL_http://www.R-project.org/. 2013.
25. Harrell J, F. E. rms: Regression Modeling Strategies. R package version 5.1-4. https://CRAN.R-project.org/package=rms. 2019.
26. Venkatesh B, Cohen J. Hydrocortisone in Vasodilatory Shock. Crit Care Clin 2019; 35: 263-275.
27. Annane D, Renault A, Brun-Buisson C, Megarbane B, Quenot J P, Siami S, Cariou A, Forceville X, Schwebel C, Martin C, Timsit J F, Misset B, Ali Benali M, Colin G, Souweine B, Asehnoune K, Mercier E, Chimot L, Charpentier C, Francois B, Boulain T, Petitpas F, Constantin J M, Dhonneur G, Baudin F, Combes A, Bohe J, Loriferne J F, Amathieu R, Cook F, Slama M, Leroy O, Capellier G, Dargent A, Hissem T, Maxime V, Bellissant E, Network C-T. Hydrocortisone plus Fludrocortisone for Adults with Septic Shock. N Eng J Med 2018; 378: 809-818.
28. Annane D, Sebille V, Charpentier C, Bollaert P E, Francois B, Korach J M, Capellier G, Cohen Y, Azoulay E, Troche G, Chaumet-Riffaud P, Bellissant E. Effect of treatment with low doses of hydrocortisone and fludrocortisone on mortality in patients with septic shock. JAMA 2002; 288: 862-871.
29. Sprung C L, Annane D, Keh D, Moreno R, Singer M, Freivogel K, Weiss Y G, Benbenishty J, Kalenka A, Forst H, Laterre P F, Reinhart K, Cuthbertson B H, Payen D, Briegel J. Hydrocortisone therapy for patients with septic shock. N Eng J Med 2008; 358: 111-124.
30. Venkatesh B, Finfer S, Cohen J, Rajbhandari D, Arabi Y, Bellomo R, Billot L, Correa M, Glass P, Harward M, Joyce C, Li Q, McArthur C, Perner A, Rhodes A, Thompson K, Webb S, Myburgh J, Investigators A T, the Australian-New Zealand Intensive Care Society Clinical Trials G. Adjunctive Glucocorticoid Therapy in Patients with Septic Shock. New Eng J Med 2018; 378: 797-808.
31. Menon K, McNally D, Choong K, Sampson M. A systematic review and meta-analysis on the effect of steroids in pediatric shock. Pediatr Crit Care Med 2013; 14: 474-480.
32. Antcliffe D B, Gordon A C. Why Understanding Sepsis Endotypes Is Important for Steroid Trials in Septic Shock. Critical care medicine 2019; 47: 1782-1784.
33. Cohen J, Blumenthal A, Cuellar-Partida G, Evans D M, Finfer S, Li Q, Ljungberg J, Myburgh J, Peach E, Powell J, Rajbhandari D, Rhodes A, Senabouth A, Venkatesh B. The relationship between adrenocortical candidate gene expression and clinical response to hydrocortisone in patients with septic shock. Intensive Care Med 2021; 47: 974-983.
34. Marik P E. The role of glucocorticoids as adjunctive treatment for sepsis in the modern era. Lancet Respir Med 2018; 6: 793-800.
35. Sweeney T E, Wong H R. Transcriptional markers in response to hydrocortisone in sepsis in ADRENAL: a step toward precision medicine. Intensive Care Med 489 2021; 47: 1011-1013.
36. Wong H R, Salisbury S, Xiao Q, Cvijanovich N Z, Hall M, Allen G L, Thomas N J, Freishtat R J, Anas N, Meyer K, Checchia P A, Lin R, Shanley T P, Bigham M T, Sen A, Nowak J, Quasney M, Henricksen J W, Chopra A, Banschbach S, Beckman E, Harmon K, Lahni P, Lindsell C J. The pediatric sepsis biomarker risk model. Crit Care 2012, 16:R174.
37. Wong H R, Weiss S L, Giuliano J S, Jr., Wainwright M S, Cvijanovich N Z, Thomas N J, Allen G L, Anas N, Bigham M T, Hall M, Freishtat R J, Sen A, Meyer K, Checchia P A, Shanley T P, Nowak J, Quasney M, Chopra A, Fitzgerald J C, Gedeit R, Banschbach S, Beckman E, Lahni P, Hart K, Lindsell C J. Testing the prognostic accuracy of the updated pediatric sepsis biomarker risk model. PLOS One 2014, 9:e86242.

What is claimed is:

1. A method of classifying a patient with septic shock as high risk of adverse outcome and/or mortality or other than high risk of adverse outcome and/or mortality, the method comprising:
obtaining a sample from a pediatric patient with septic shock at a first time point;
analyzing the sample to determine the expression levels of 100 biomarkers to generate a gene expression mosaic for the patient;
determining a Z value for the patient according to Equation 1:
$$Z = \vert {Ref}_{A} - {Patient}_{i} \vert - \vert {Ref}_{B} - {Patient}_{i} \vert . \qquad Equation 1$$
wherein Ref_{A} is a reference mosaic for endotype A, Ref_{B} is a reference mosaic for endotype B, and Patienti is the gene expression reference mosaic for the patient; classifying the patient as high risk or other than high risk, wherein a classification of high risk comprises a Z value below a cutoff Z value, and wherein a classification of other than high risk comprises a Z value of the cutoff Z value or greater; and
wherein the 100 biomarkers comprise:
APAF1; ARPC5; ASAHI; ATP2B2; BCL6; BMPR2; BTK; CAMK2D; CAMK2G; CAMK4; CASP1; CASP2; CASP4; CASP8; CD247; CD3E; CD3G; CD79A; CREB1; CREB5; CSNKIA1; CTNNB1; DAPP1; DBT; EP300; FAS; FCGR2A; FCGR2C; FYN; GK; GNAI3; HDAC4; HLA-DMA; HLA-DOA; ICAM3; ILIA; INPP5D; ITGAM; ITGAV; ITGAX; JAK1; JAK2; KAT2B; LAT2; LYN; MAP2K4; MAP3K1; MAP3K3; MAP3K5; MAP3K7; MAP4K1; MAP4K4; MAPK1; MAPK14; MDH1; MKNK1; NCOA2; NCR3; NFATC1; PAK2; PDPR; PIAS1; PIK3C2A; PIK3C3; PIK3CA; PIK3CD; PIK3R1; PLCG1; POU2F2; PPP1R12A; PPP2R2A; PPP2R5C; PRKARIA; PRKCB; PSMB7; PTEN; PTPRC; RAF1; RHOT1; ROCK1; SEMA4F; SEMA6B; SMAD4; SOS1; SOS2; SP1; TAF11; TBK1; TGFBR1; TLE4; TLR1; TLR2; TLR8; TNFSF10; TRA@; TYROBP; UBE3A; USP48; ZAP70; and ZDHHC17.

2. The method of claim 1, wherein the cutoff Z value is determined by modeling an interaction between the Z value and receipt of corticosteroids using logistic regression.

3. The method of claim 2, comprising fitting the Z value using cubic splines.

4. The method of any preceding claim, wherein the cutoff Z value is about 15.

5. The method of any preceding claim, wherein the difference between the gene expression reference mosaic for the patient and the reference mosaic is calculated by pixel-to-pixel intensity difference.

6. The method of claim 1, wherein biomarker expression levels are determined by mRNA quantification.

7. The method of claim 6, wherein biomarker expression levels are determined by normalized mRNA counts and/or by cycle threshold (CT) values.

8. The method of claim 1, wherein the classification is combined with one or more patient demographic data and/or clinical characteristics and/or results from other tests or indicia of septic shock.

9. The method of claim 8, wherein the patient demographic data and/or clinical characteristics and/or results from other tests or indicia of septic shock comprise at least one selected from the group consisting of the septic shock causative organism, the presence or absence or chronic disease, and/or the age, gender, race, and/or co-morbidities of the patient.

10. The method of claim 1, wherein the classification is combined with one or more additional population-based risk scores.

11. The method of claim 10, wherein the one or more population-based risk scores comprises at least one selected from the group consisting of Pediatric Sepsis Biomarker Risk Model (PERSEVERE), Pediatric Risk of Mortality (PRISM), Pediatric Index of Mortality (PIM), and/or Pediatric Logistic Organ Dysfunction (PELOD).

12. The method of claim 1, wherein the sample is obtained within the first hour of presentation with septic shock.

13. The method of claim 1, wherein the sample is obtained within the first 48 hours of presentation with septic shock.

14. The method of claim 1, further comprising administering a treatment comprising one or more corticosteroid to a patient that is not high risk, or administering a treatment comprising one or more therapy excluding a corticosteroid to a patient that is classified as high risk, to provide a method of treating a pediatric patient with septic shock.

15. The method of claim 14, wherein one or more high risk therapy is administered to a patient classified as high risk.

16. The method of claim 15, wherein the one or more high risk therapy comprises at least one selected from the group consisting of immune enhancing therapy, extracorporeal membrane oxygenation/life support, plasmapheresis, pulmonary artery catheterization, and/or high volume continuous hemofiltration.

17. The method of claim 16, wherein the immune enhancing therapy comprises administration of GMCSF, interleukin-7, and/or anti-PD-1.

18. The method of claim 14, comprising improving an outcome in a pediatric patient with septic shock.

19. The method of claim 14, further comprising:
obtaining a second sample from the treated patient at a second time point;
analyzing the second sample to determine the expression levels of the 100 biomarkers listed in claim 1 to generate a gene expression mosaic for the patient;
determining the patient's Z value; and
maintaining the treatment being administered if the patient's high risk classification has not changed, or changing the treatment being administered if the patient's high risk classification has changed.

20. The method of claim 18, wherein the second time point is at least 18 hours after the first time point.

21. The method of claim 20, wherein the second time point is in the range of 24 to 96 hours, or longer, after the first time point.

22. The method of claim 21, wherein the second time point is about 1 day, 2 days, 3 days, or longer, after the first time point.

23. The method of claim 22, wherein the second time point is about 2 days after the first time point.

24. The method of claim 23, wherein the first time point is at day 1, wherein day 1 is within 24 hours of a septic shock diagnosis, and the second time point is at day 3.

25. The method of claim 20, wherein a patient classified as high risk after the second time point is administered one or more high risk therapy.

26. The method of claim 25, wherein the one or more high risk therapy comprises at least one selected from the group consisting of immune enhancing therapy, extracorporeal membrane oxygenation/life support, plasmapheresis, pulmonary artery catheterization, and/or high volume continuous hemofiltration.

27. The method of claim 26, wherein the one or more high risk therapy comprises an immune enhancing therapy.

28. The method of claim 20, wherein a patient not classified as high risk after the second time point is administered a treatment comprising one or more corticosteroid.

29. The method of claim 28, wherein the patient classified as high risk and administered one or more high risk therapy after the first time point is not classified as high risk after the second time point.

30. The method of any preceding claim, wherein the patient is a pediatric patient.
